(19) United States
(12) Patent Application Publication
(10) Pub. No.: US 2004/0151326 A1
(43) Pub. Date: Aug. 5, 2004

(54) Active diffracted sound control apparatus

(76) Inventors: Osamu Nishimura, Kawasaki-shi (JP); Akihiko Enamito, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/717,554

(22) Filed: Nov. 21, 2003

(30) Foreign Application Priority Data
Nov. 22, 2002 (JP) 2002-339829

(51) Int. Cl. A61F 11/06; G10K 11/16; H03B 29/00

(52) U.S. Cl. 381/71.2

(57) ABSTRACT

An active diffracted sound control apparatus comprises a reference microphone for measuring the noise information on a noise source side of a sound insulating wall, a first error microphone for measuring the noise information on a noise receiving side of the sound insulating wall, a speaker arranged in the neighborhood of the sound insulating wall K for generating a control sound to reduce the object sound at a sound receiving point, and a control unit for controlling the output of the speaker based on the outputs of the reference microphone and the first error microphone. The control unit is operated based on a first sound transmission characteristic between the first error microphone and the second error microphone for the noise source and a second sound transmission characteristic between the first error microphone and the second error microphone for the speaker.

### CROSS-REFERENCE TO RELATED APPLICATIONS

This application is based upon and claims the benefit of priority from the prior Japanese Patent Application No. 2002-339829, filed Nov. 22, 2002, the entire contents of which are incorporated herein by reference.

### BACKGROUND OF THE INVENTION

1. Field of the Invention

The present invention relates to an active diffracted sound control apparatus for reducing the sound to be controlled (hereinafter referred to as the object sound) such as noises arriving by diffracting a wall built to shut out or insulate the sound, or more in particular to an active diffracted sound control apparatus having a small space of installation.

2. Description of the Related Art

To shut out or insulate the sound by installing a wall between a noise source area generating a noise (the sound to be controlled) and a sound receiving area where the noise is desirably reduced is a sound-insulating technique generally used as a measure to shut out or insulate noises from buildings or roads. The active noise control technique for reducing noises by the sound generated from a speaker (control sound source) is known to reduce the noises by feedback control.

In spite of the provision of a sound insulating wall, some noises override the wall by diffraction. In view of this, a method has been generally proposed which uses the active noise control technique. According to this method, a noise source measuring sensor and a control sound source are installed on the noise source side of the noise insulating wall and the sound pressure level detected by the noise source measuring sensor is reduced by use of the noise source measuring sensor and the control sound source thereby to reduce the diffracted sound on the sound-receiving area side of the wall. The use of this method, however, requires installation of the noise source measuring sensor and the control sound source at a place distant from the noise insulating wall, which in turn requires a space for installing the noise source measuring sensor and the control sound source between a noise source and the sound insulating wall.

The reason is as follows. FIGS. 8A to 8D are diagrams showing the result of application of the active noise control technique described above. FIG. 8A is a diagram for explaining the relative positions of error microphones. The relationship between the positions of the error microphones and the noise reducing effect is shown in FIGS. 8A to 8D. This relationship represents the distribution of sound pressure of 0 to 1000 Hz band in the Y-Z plane in FIG. 8A. The well-known filtered-X algorithm can be used as an active noise control method.

In addition to the noise reduction by the diffraction effect of the wall, the noise is reduced by being offset by the sound produced from the control sound source. The difference between the result Q1 obtained by the error microphone M1 near the sound insulating wall and the result Q2 obtained by use of the error microphone M2 comparatively far from the sound insulating wall is indicated by Q3. The fact that all Q3 are positive apparently indicates that the noise reduction area is widened and the noise reduction effect is improved with the increase in the distance of the error microphones from the sound insulating wall.

This is also evident from the general attenuation characteristics of the sound. The distance from the speaker installed at the upper end of the wall to the error microphones is extremely short as compared with the distance between the noise source some distance away from the wall and the error microphones. The sound power attenuates in inverse proportion to the square of the distance, and the gradient of the attenuation curve is so steep at a point short in distance. Even in the case where the gain from the noise source and the gain from the control sound source are combined at this point, the power difference in the area beyond the particular point is so great that the noise is reduced only at the position of the error microphones, and the area where the noise is reduced is very limited.

FIGS. 9A and 9B are diagrams for explaining the above-mentioned phenomenon briefly. Specifically, as shown in FIG. 9A, in the case where the gain curve of the noise source and the gain curve of the control sound source intersect at a place near the sound insulating wall, the sound power difference after the intersection increases. As shown in FIG. 9B, on the other hand, in the case where the gain curve of the noise source and the gain curve of the control sound source intersect at a place far from the sound insulating wall, the sound power difference decreases beyond the intersection. This indicates that the error microphones are preferably installed distantly from the sound insulating wall to improve the noise reducing effect in an area where the diffracted sound reaches.

Various methods have been conceived other than the active diffracted sound control method described above. In a method for suppressing the diffracted sound with a speaker installed at the upper end of a sound insulating wall, for example, the size and direction of the speaker (control sound source) installed at the upper end of the wall are limited. The description of this method, however, contains no specific way of processing and outputting a signal nor the place in the sound receiving area of the speaker to reduce the noise.

In a method using the control sound source arranged at the upper end of the sound insulating wall with the observed noise phase inverted and output, on the other hand, noise source measuring device and the control sound source are integrated, and the measured noise phase is inverted and output to the control sound source. This method fails to reveal the manner in which the phase of the sound transmission characteristic between the noise source measuring device and the control sound source is inverted.

Further, a method with the speaker installed in the neighborhood of the surface of the noise source but not at the upper end of the sound insulating wall includes the description of how the speaker is installed for reducing the direct sound as well as the diffracted sound and the calculation of the control output. This method, however, fails to reveal the relationship between the area where the noise on the sound receiving side is desirably reduced and the noise measuring point.

The above-mentioned active diffracted sound control apparatuses pose the following-described problem. Specifically, in the conventional active noise control technique for reducing the noise arriving by diffraction, the spatial limitation is imposed in which the control sound source is required to be installed at a point distant from the wall on the noise source side. In many cases, however, no such installation space is available on the two sides of the sound insulating wall. In the case where the diffracted sound control apparatus is installed additionally on the existing sound insulating wall, on the other hand, a new installation space and a long support for the error microphones are required.

Even in the case where the speaker providing the control sound source is arranged at the upper end of the sound insulating wall, how the speaker is controlled is not made apparent. Further, in the case where the noise is reduced at the noise measuring point near the sound insulating wall, it is not apparent how the noise reduction area is formed on the sound receiving side of the sound insulating wall, thereby failing to define the effective range.

### SUMMARY OF THE INVENTION

Accordingly, it is an object of the present invention to provide an active diffracted sound control apparatus which can reduce the sound pressure level of the object sound at the sound receiving point simply by installing a reference microphone, an error microphone, a speaker and the like in the neighborhood of a wall body without arranging any error microphone at the sound receiving point distant from the wall body.

In order to solve the problem and to achieve the object described above, according to this invention, there is provided an active diffracted sound control apparatus mounted on a wall body between an object sound source area and a sound receiving area for reducing the object sound generated in the object sound source area, comprising sound source measuring device arranged on the object sound source area side of the wall body for measuring the object sound source information in the neighborhood of the wall body, direct object sound measuring device arranged on the sound receiving area side of the wall body for measuring the object sound information in the neighborhood of the wall body, a control sound source arranged in the neighborhood of the wall body for generating a control sound to reduce the object sound at a virtual object sound measuring point in the sound receiving area, and object sound control device for controlling the output of the control sound based on the output of the object sound measuring device, wherein the object sound control device operates based on a first sound transmission characteristic between the direct object sound measuring point and the virtual object sound measuring point for the object sound source and a second sound transmission characteristic between the direct object sound measuring point and the virtual object sound measuring point for the control sound source.

### BRIEF DESCRIPTION OF THE DRAWINGS

FIG. 1A is a perspective view schematically showing an active diffracted sound control apparatus mounted on a sound insulating wall according to a first embodiment of the invention;

FIG. 1B is a side view schematically showing an active diffracted sound control apparatus mounted on a sound insulating wall according to the same embodiment;

FIG. 2 is a schematic diagram for explaining the relative positions of the sound insulating wall, a noise source, a sound receiving point and the active diffracted sound control apparatus;

FIG. 3 is a diagram for explaining the transmission characteristics of the noise and the control sound from the noise source to a second error microphone;

FIG. 4 is a diagram showing an example of the control result obtained by the active diffracted sound control apparatus;

FIG. 5A is a diagram showing the relationship between the relative positions of the sound insulating wall and the first and second error microphones on the one hand and the amount of noise reduction on the other hand;

FIG. 5B is a diagram showing the relationship between the relative positions of the sound insulating wall and the first and second error microphones on the one hand and the gain on the other hand;

FIG. 6 is a diagram showing the relationship between the identification section and the control section of the active diffracted sound control apparatus;

FIG. 7 is a perspective view showing an example of application of the active diffracted sound control apparatus to a building opening;

FIG. 8A is a diagram for explaining the relative positions of the error microphones;

FIGS. 8B to 8D are diagram for explaining the relationship between the positions of the error microphones and the noise reduction effect; and

FIGS. 9A and 9B are diagrams for explaining the relationship between noise level and distance.

### DETAILED DESCRIPTION OF THE INVENTION

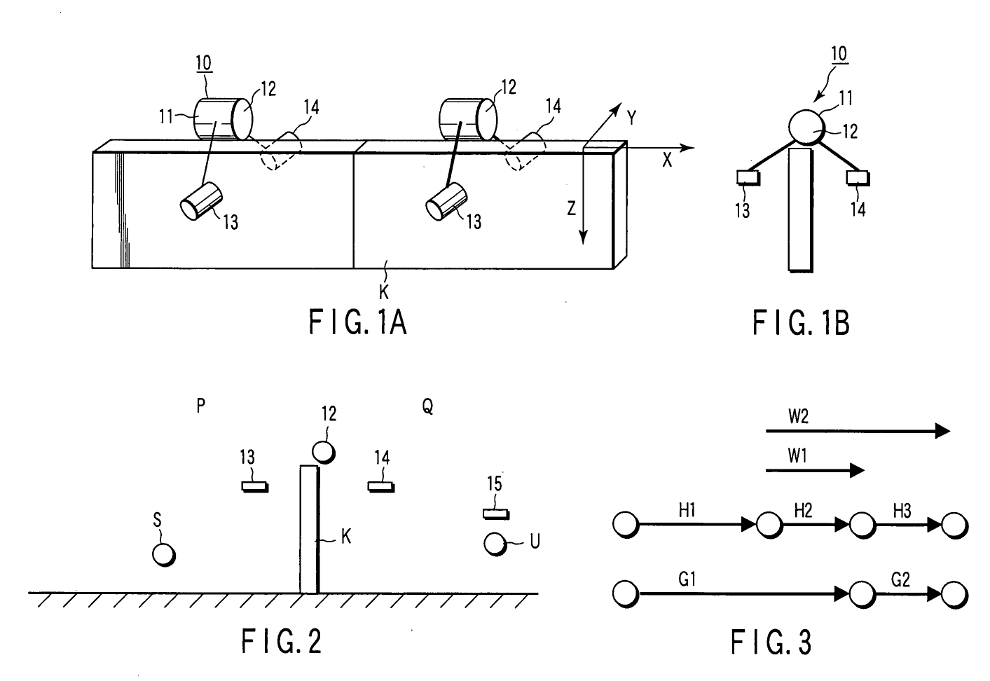

FIG. 1A is a perspective view showing an outline of an active diffracted sound control apparatus 10 according to a first embodiment of the invention mounted on a sound insulating wall K. FIG. 1B is a side view showing an outline of the active diffracted sound control apparatus 10 according to the same embodiment mounted on the sound insulating wall K.

FIG. 2 is a schematic diagram for explaining the relative positions of the sound insulating wall K, a noise source S and the active diffracted sound control apparatus 10. In FIG. 2, reference character S designates the noise source, character U a sound receiving point, character P a noise area, and character Q a noise receiving area.

The active diffracted sound control apparatus 10 comprises a body 11 mounted on the sound insulating wall K, a speaker (control sound source) 12 arranged on the body 11, a reference microphone (sound source measuring device) 13 connected with the body 11 through an arm 11a and arranged in the noise area P for directly measuring the noise from the noise source S, a first error microphone (direct object sound measuring device) 14 connected with the body 11 through an arm 11b and arranged in the sound receiving area Q for directly measuring the noise from the noise source S, and a control unit (object sound control device) 20 arranged in the body 11 for calculating the output level of the control sound from the speaker 12 based on the input from the reference microphone 13 and the first error microphone 14.

An arm 11b for supporting the first error microphone 14 is arranged in such a position that the distance from the sound insulating wall K to the first error microphone 14 is within the distance of the shortest wavelength of the frequency of the object sound. In the case where the frequency of the object sound is 1000 Hz at the maximum, for example, the shortest wavelength is about 34 cm. Suppose an arm 11a for supporting the reference microphone 13 is of about the same length, then the reference microphone 13, the speaker 12 and the first error microphone 14 are all installed in the neighborhood (within several tens of cm) of the sound insulating wall K and can be integrally arranged.

The control unit 20 uses the transmission characteristics H3 and G2 when calculating the output level of the control sound from the speaker 12 based on the input from the reference microphone 13 and the first error microphone 14. Specifically, before the object sound is controlled by the active diffracted sound control apparatus 10, the second error microphone 15 is arranged at the sound receiving point U so that the transmission characteristics H3 and G2 are acquired and stored in the control unit 20.

The transmission characteristics H3 and G2 thus stored and the information from the reference microphone 13 and the first error microphone 14 obtained from the time point when the control operation is started are used for calculation thereby to determine the output of the speaker 12.

The principle for control operation of the control unit 20 is explained. FIG. 3 is a diagram for explaining the transmission characteristics of the noise and the control sound from the noise source S to the second error microphone 15. Character H1 designates the transmission characteristic of the noise from the noise source S to the speaker 12, character H2 the transmission characteristic of the noise from the speaker 12 to the first error microphone 14, and character H3 the transmission characteristic of the noise from the first error microphone 14 to the second error microphone 15. On the other hand, character G1 designates the transmission characteristic of the control sound from noise source S to the first error microphone 14, and character G2 the transmission characteristic of the control sound from the first error microphone 14 to the second error microphone 15.

Suppose the active diffracted sound control operation is performed in such a manner as to reduce the sound pressure level of the noise in the first error microphone 14. The transmission characteristic W1 can be acquired by the control unit 20. What is actually required to be reduced, however, is the sound pressure level at the sound receiving point U. Unless the transmission characteristic W2 including the transmission characteristic H3 up to the second error microphone 15 is acquired, therefore, the noise at the sound receiving point U where the second error microphone 15 is installed is not reduced. In view of this, the transmission characteristics H3 and G2 of the sound between the first error microphone 14 and the second error microphone 15 are measured and held as data in advance, and using these data and the transmission characteristic W1, the transmission characteristic W2 is determined from the equation below.

W₂=W₁H₃⁻¹·G₂ (1)

The use of this transmission characteristic W2 makes it possible to control the noise in the same manner as if the second error microphone 15 exists at the sound receiving point U.

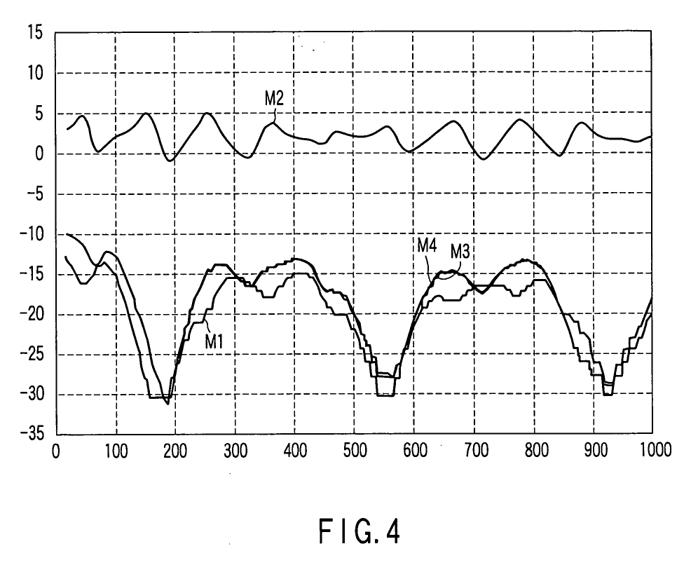

FIG. 4 is a diagram showing an example of the result obtained by the method described above. Character M1 designates the transmission characteristic W1, character M2 the ratio of the transmission characteristic G2 to the transmission characteristic H3, character M3 the transmission characteristic W2 calculated from M1 and M2, and character M4 the transmission characteristic W2 actually calculated using the second error microphone 15. Since M3 substantially is equal to M4, the effectiveness of this method is understood.

Now, the arrangement of the first error microphone 14 and the second error microphone 15, i.e. the distance at which the first error microphone 14 and the second error microphone 15 are required to be located from the sound insulating wall K are explained. As long as the transmission characteristics H3 and G2 are measured and held as data in advance, the noise can be controlled, as described above. With the advance of time, however, the transmission characteristics H3 and G2 may deviate from the data measured in advance. In the case where the distance between the first error microphone 14 and the second error microphone 15 is extremely large, for example, the transmission characteristics H3 and G2 are expected to vary greatly with temperature change or the like, thereby probably adversely affecting the noise reduction effect.

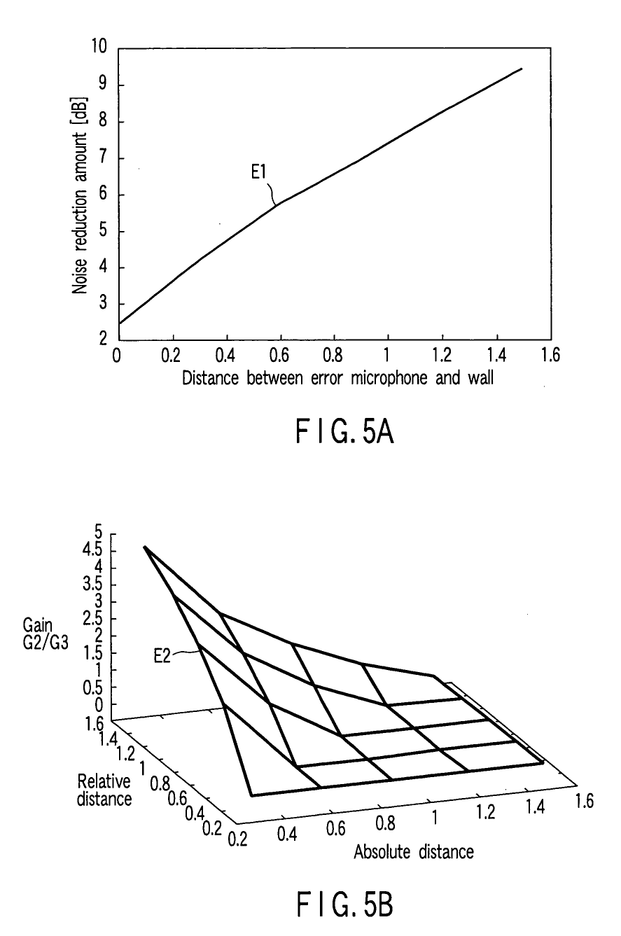

FIG. 5A shows the relationship between the absolute distance from the second error microphone 15 to the sound insulating wall K and the noise reduction effect for the object sound up to 1000 Hz. As seen from the graph, the larger the distance of the second error microphone 15 from the sound insulating wall K, the greater the noise reduction effect. For practical applications, however, the noise is desirably reduced by about 3 to 4 dB. Understandably, therefore, the second error microphone 15 is desirably located at least 0.3 m away, i.e. at a point more distant than the shortest wavelength λ of the frequency of the object sound from the sound insulating wall K. In view of the fact that the second error microphone 15 is located at a point more distant than the shortest wavelength λ from the upper end portion of the wall, however, the first error microphone 14 is required to be arranged within the distance of the shortest wavelength λ from the upper end portion of the sound insulating wall K.

FIG. 5B is a graph showing the relationship between the absolute distance from the first error microphone 14 to the sound insulating wall K, the relative distance from the first error microphone 14 to the second error microphone 15, and the gain, i.e. the ratio of the transmission characteristic G2 to the transmission characteristic H3 for the object sound up to 1000 Hz. In FIG. 5B, character E2 designates the average gain value. The larger the relative distance between the first error microphone 14 and the second error microphone 15, the higher the sensitivity to the variation of the transmission characteristics H3 and G2. Therefore, the average gain value is desirably as small as possible (about not more than 2 dB). This graph shows that in the case where the first error microphone 14 is arranged within the distance of the shortest wavelength λ of the object sound frequency from the sound insulating wall K, the relative distance between the first error microphone 14 and the second error microphone 15 is desirably not more than double the wavelength λ.

The fact described above indicates that one wavelength is equal to 34 cm in the case where the frequency of the object sound is not higher than 1000 Hz, for example. As a result, the first error microphone 14 is installed at about 30 cm from the sound insulating wall K, thereby facilitating the production of an integrated type of the apparatus. Further, the noise control operation is possible in the same manner as if the second error microphone 15 exists at a point two wavelengths (70 cm) away from the first error microphone 14. This difference of several tens of cm brings about a large difference in the noise reduction area and the noise reduction effect.

As described above, even in the case where the second error microphone 15 is not actually arranged at the sound receiving point U distant from the sound insulating wall K and desirably lower in noise level, the use of the active diffracted sound control apparatus 10 according to this embodiment can reduce the noise level at the sound receiving point U. Specifically, simply by installing the reference microphone 13, the speaker 12 and the first error microphone 14 in the neighborhood (about several tens of cm) of the sound insulating wall K, the noise level at the sound receiving point U can be reduced. This increases the freedom of installation. Further, an integrated structure of the reference microphone 13, the speaker 12 and the first error microphone 14, as shown in FIG. 1, makes possible the installation at the upper end portion of the sound insulating wall K or in a simple way, thereby contributing to an improved installation convenience.

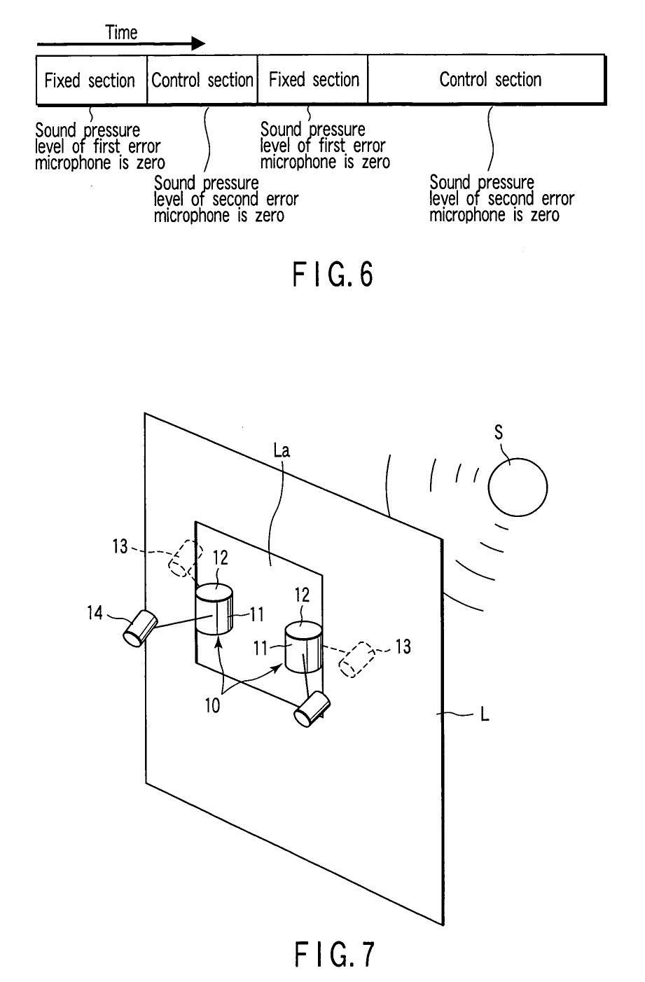

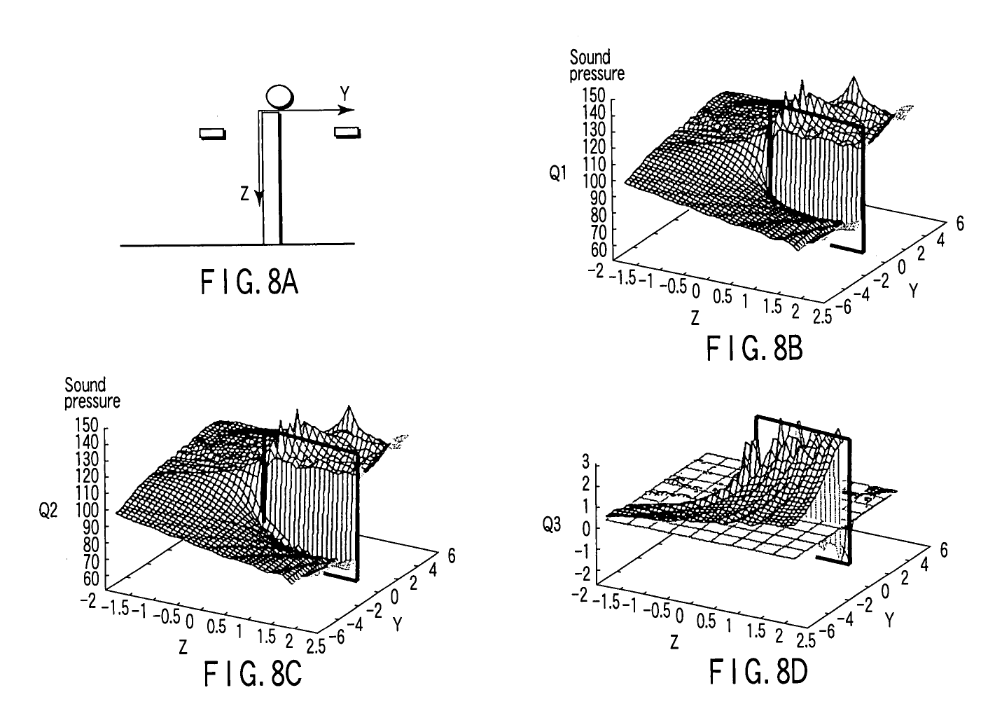

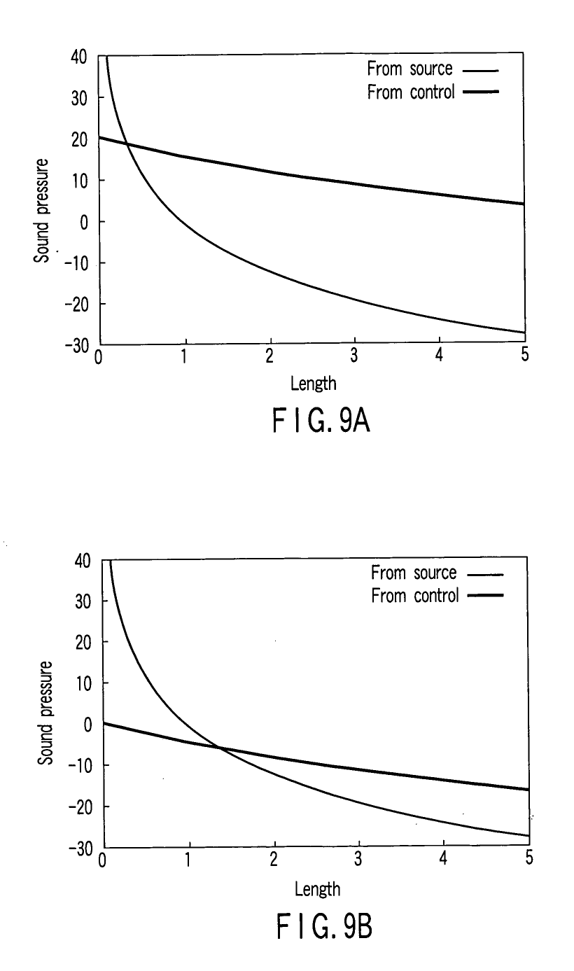

FIG. 6 is a diagram for explaining an example of application of the active diffracted sound control apparatus 10 described above. Specifically, the actual transmission characteristic W1 of the active diffracted sound control apparatus and the transmission characteristic W2 used for actually reducing the noise are required to be acquired under different circumstances. Therefore, an identification section for acquiring the transmission characteristic W1 to reduce the sound pressure level of the first error microphone 14 to zero is required to be set differently from a control section for controlling the sound pressure level of the second error microphone 15 to zero using the transmission characteristic W2. This identification section should be sufficiently long enough to adjust the transmission characteristic W1. Also, unless the environment or the transmission characteristic undergoes a considerable change, it is enough to acquire the transmission characteristic W1 once first and calculate the transmission characteristic W2.

FIG. 7 shows an example of application in which the active diffracted sound control apparatuses 10 described above are used for an opening La of a wall member L of a building or the like. The active diffracted sound control apparatus 10 is applicable not only to the sound insulating wall or the sound proofing wall for roads or the like, but also to any other wall bodies with equal effect. In this way, the noise leaking out from the opening La of a building can be reduced.

The present invention is not confined to the embodiments described above. Although the foregoing description deals with an integrated type of active diffracted sound control apparatus, the invention is applicable to a separated type of active diffracted sound control apparatus with equal effect. Also, the second error microphone, which is removed after acquiring the transmission characteristics H3 and G2 in the foregoing case, may actually be installed so long as the installation conditions are met. Also, so long as predetermined conditions are met such as in the case where the transmission characteristics H3 and G2 are known, for example, the second error microphone may not be installed. Further, the active diffracted sound control apparatus according to the invention is of course variously modifiable without departing from the scope and spirit of the invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An active diffracted sound control apparatus comprising:
sound source measuring device arranged on the object sound source area side of a wall body arranged between the object sound source area and a sound receiving area for measuring the object sound information in the neighborhood of the wall body;
direct object sound measuring device arranged on the sound receiving area side of the wall body for measuring the object sound information in the neighborhood of the wall body;
a control sound source arranged in the neighborhood of the wall body for generating a control sound to reduce the object sound at a virtual object sound measuring point in the sound receiving area; and
object sound control device for controlling the output of the control sound based on the output of the object sound measuring device;
wherein the object sound control device is operated on the basis of a first sound transmission characteristic between the direct object sound measuring point for the object sound source and the virtual object sound measuring point and
a second sound transmission characteristic between the direct object sound measuring point for the control sound source and the virtual object sound measuring point.

2. An active diffracted sound control apparatus according to claim 1,
wherein the object sound control device alternates between a control section for generating the control sound and an identification section for obtaining a third sound transmission characteristic between the object sound source measuring device and the direct sound measuring device.

3. An active diffracted sound control apparatus according to claim 1,
wherein the sound source measuring device, the direct object sound measuring device and the control sound source are configured integrally and installed removably on the wall body.

4. An active diffracted sound control apparatus according to claim 1,
wherein the control sound source is arranged at the upper end portion of the wall body, and
wherein the direct object sound measuring device is arranged within the distance of the shortest wavelength of the frequency of the object sound from the upper end portion of the wall body.

5. An active diffracted sound controller comprising:
sound source measuring device arranged on that part of a wall body having an opening between a controlled sound source area and a sound receiving area which is nearer to the controlled sound source area for measuring the controlled sound information in the neighborhood of the opening;
direct controlled sound measuring device arranged on the part of the opening nearer to the sound receiving area for measuring the controlled sound information in the neighborhood of the opening;
a control sound source arranged in the neighborhood of the opening for generating a control sound for reducing the controlled sound at a virtual controlled sound measuring position in the sound receiving area; and
controlled sound control device for controlling the output of the control sound based on the output of the controlled sound measuring device;
wherein the controlled sound control device is operated based on a first sound transmission characteristic of the controlled sound source between the direct controlled sound measuring position and the virtual controlled sound measuring position, and a second sound transmission characteristic of the control sound source between the direct controlled sound measuring position and the virtual controlled sound measuring position.

6. An active diffracted sound controller according to claim 5,
wherein the controlled sound control device is operated alternately in a control section for generating a control sound and an identification section for producing a third sound transmission characteristic of the controlled sound source between the controlled sound source measuring device and the direct sound measuring device.

7. An active diffracted sound controller according to claim 5,
wherein the sound source measuring device, the direct controlled sound measuring device and the control sound source are configured integrally and adapted to be arranged removably on the opening.

8. An active diffracted sound controller according to claim 5,
wherein the control sound source is arranged at the upper end portion of the wall body; and
wherein the direct controlled sound measuring device is arranged within the distance of the shortest wavelength of the frequency of the controlled sound from the edge portion of the opening.

9. An active diffracted sound control method comprising:
a sound source measuring the controlled sound information at a sound source measuring position in the neighborhood of that part of a wall body which is nearer to the controlled sound source area, the wall body being arranged between the controlled sound source area and the sound receiving area;
a direct controlled sound measuring the controlled sound information at a direct controlled sound measuring position in the neighborhood of that part of the wall body which is nearer to the sound receiving area;
a control sound generating a control sound, at a control sound generating position in the neighborhood of the wall body, for reducing the controlled sound at a virtual controlled sound measuring position in the sound receiving area; and
a controlled sound controlling the output of the control sound based on the output from the information of the controlled sound measured in the direct controlled sound measuring ;
wherein the controlled sound controlling is operated based on a first sound transmission characteristic of the controlled sound between the direct controlled sound measuring position and the virtual controlled sound measuring position, and a second sound transmission characteristic of the control sound between the direct controlled sound measuring position and the virtual controlled sound measuring position.

10. An active diffracted sound control method according to claim 9,
wherein said controlled sound controlling is executed alternately in a control section for generating a control sound and an identification section for producing a third sound transmission characteristic of the controlled sound between the controlled sound source measuring position and the direct sound measuring position.

11. An active diffracted sound control method according to claim 9,
wherein the control sound generating position is at the upper end portion of the wall body, and
wherein the direct controlled sound measuring position is within the distance of the shortest wavelength of the frequency of the controlled sound from the upper end portion of the wall body.

12. An active diffracted sound control method comprising:
a first sound transmission characteristic measuring a first sound transmission characteristic of the controlled sound between the direct controlled sound measuring position in the neighborhood of the part of the wall body nearer to the sound receiving area and the virtual controlled sound measuring position on the part of the wall body nearer to the sound receiving area, the wall body being arranged between the controlled sound source area and the sound receiving area;
a second sound transmission characteristic measuring a second sound transmission characteristic of the control sound between the direct controlled sound measuring position and the virtual controlled sound measuring position;
a sound source measuring the controlled sound information at a sound source measuring position in the neighborhood of the part of the wall body nearer to the controlled sound source area;
a direct controlled sound measuring the controlled sound information at the direct controlled sound measuring position;
a control sound generating a control sound at a control sound generating position in the neighborhood of the wall body for reducing the controlled sound at the virtual controlled sound measuring position; and
a controlled sound controlling the output of the control sound based on the output from the information of the controlled sound measured in the direct controlled sound measuring, the first sound transmission characteristic and the second sound transmission characteristic.

13. An active diffracted sound control method according to claim 12,
wherein the controlled sound controlling is executed alternately between a control section for generating the control sound and an identification section for producing a third sound transmission characteristic of the controlled sound between the controlled sound source measuring position and the direct sound measuring position.

14. An active diffracted sound control method according to claim 12,
wherein the control sound generating position is at the upper end portion of the wall body, and the direct controlled sound measuring position is within the distance of the shortest wavelength of the frequency of the controlled sound from the upper end portion of the wall body.
